(19) United States
(12) Patent Application Publication
(10) Pub. No.: US 2011/0263659 A1
(43) Pub. Date: Oct. 27, 2011

(54) Analogs of 1-Methylnicotinamide

(75) Inventor: Alexander Krantz, Boston, MA (US)

(73) Assignee: Cortria Corporation, Needham, MA (US)

(21) Appl. No.: 12/991,056

(22) PCT Filed: May 5, 2009
(86) PCT No.: PCT/US09/42906

§ 371 Date: Jan. 26, 2011

Related U.S. Application Data
(60) Provisional application No. 61/126,642, filed on May 6, 2008.

(51) Int. Cl. A61K 31/4406; A61P 9/00; A61P 3/10; A61P 35/00; A61P 19/10; A61P 3/04; A61P 17/00; A61P 9/10; A61P 9/04; A61P 25/28; A61P 9/12; A61P 11/16; A61P 15/10; A61P 1/00; C07D 213/56

(52) U.S. Cl. 514/355; 546/316

(57) ABSTRACT

The present invention is directed to derivatives and analogs of 1-methyl-nicotinamide useful for elevating the blood levels of 1-methylnicotinamide or deuterated species in a subject.

### BACKGROUND OF THE INVENTION

1-Methylnicotinamide (1-MNA) exhibits diverse biological activities, including anti-inflammatory properties: Biedron et al., 2008, “1-Methylnicotinamide and nicotinamide: two related anti-inflammatory agents that differentially affect the functions of activated macrophages”, J. Arch. Immunol. Ther. Exp. (Warsz) 56(2):127-34, and references therein; Gebicki et al., 2003, “1-Methylnicotinamide: a potent anti-inflammatory agent of vitamin origin”, Pol. J. Pharmacol. 55(1):109-12.

1-MNA has been the subject of clinical trials as a dyslipidemic agent. Similarly, a variety of therapeutic indications have been proposed for this molecule on the basis of preclinical experiments, such as are disclosed in PCT/IB06/004013, PCT/EP05/050057, and U.S. patent application Ser. No. 11/715,660. Each of these applications includes the demonstration of 1-MNA's ability to stimulate the release of prostacyclin (PGI₂). See also Chlopicki et al., 2007, “1-Methylnicotinamide (MNA), a primary metabolite of nicotinamide, exerts anti-thrombotic activity mediated by a cyclooxygenase-2/prostacyclin pathway”, Br. J .Pharmacol. 152(2):230-9.

1-MNA is an organic cation that does not cross the blood brain barrier and apparently does not enter cells. Recent studies from Smolenski et al. establish that 1-MNA possesses a half life of 2-3 hours in human subjects. Given the diverse biological activities and associated utilities for 1-MNA, it is of substantial interest to develop strategies designed to increase its half life, broaden its distribution, and determine its effects intracellularly. There is thus a long felt but unmet need in the art for a longer acting formulation of 1-MNA that, for example, might provide the ability to elevate blood levels of 1-MNA and permit efficacious once-a-day dosage forms.

### SUMMARY OF THE INVENTION

The present invention is directed to 1-MNA analogs, derivatives, and deuterated species thereof that increase the half life of 1-MNA.

In one embodiment, the invention includes a compound of Formula I or Formula II:

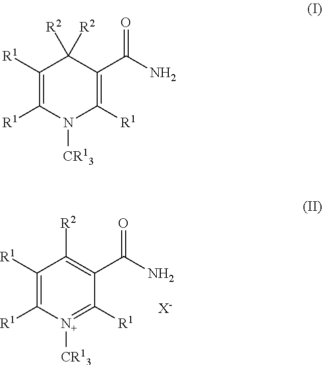

wherein

each R¹ is independently selected at each occurrence from the group consisting of H or D;

R² is independently selected at each occurrence from the group consisting of H, D, or CR¹₃, provided that where R² may be substituted on a molecule twice, both occurrences are not CR¹₃;

at least one of R¹ or R² is D; and

X⁻, when present, is a pharmaceutically acceptable counter ion.

In another embodiment, the invention comprises a method of treating or diagnosing a disease or disorder comprising administering to the subject in need thereof a therapeutically-effective amount of compound of Formula (I) or (II).

In an embodiment, the disease or disorder is heart disease, diabetes, cancer, osteoporosis, obesity, skin disorders, venous thrombosis, myocardial infarction, stroke, congestive heart failure, Alzheimer's disease, eczema, atherosclerosis, hyperlipidemia, hypertension, cerebral vasospasm, coronary vasospasm, bronchial asthma, preterm labor, erectile dysfunction, glaucoma, vascular smooth muscle cell proliferation, myocardial hypertrophy, malignoma, ischemia/reperfusion-induced injury, endothelial dysfunction, Crohn's Disease, colitis, irritable bowel disease, neurite outgrowth, Raynaud's Disease, angina, or benign prostatic hyperplasia.

In yet another embodiment, the compound of Formula (I) or (II) is administered orally, nasally, rectally, intravaginally, intravesically, parenterally, buccally, sublingually, or topically.

In another embodiment, the compound of Formula (I) or (II) is formulated using one or more pharmaceutically acceptable excipient chosen from the group consisting of starch, sugar, cellulose, diluent, granulating agent, lubricant, binder, disintegrating agent, wetting agent, emulsifier, coloring agent, release agent, coating agent, sweeting agent, flavoring agent, perfuming agent, preservative, antioxidant, plasticizer, gelling agent, thickener, hardener, setting agent, suspending agent, surfactant, humectant, carrier, and stabilizer, or a combination there.

In a further embodiment, the subject is a mammal. In yet another embodiment, the subject is human.

In still another embodiment, the pharmaceutically acceptable counter ion is selected from the group consisting of chloride, bromide, benzoate, salicylate, acetate, citrate, and lactate.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### DETAILED DESCRIPTION OF THE INVENTION

A method of increasing blood levels of 1-MNA is to administer a prodrug, such as 1,4-dihydro-1-methyl-nicotinamide: Erb et al., 1999, Biochem. Pharmacol. 57 (6):681-4. Erb et al. synthesized and administered the brain-permeable prodrug, 1,4-dihydro-1-methylnicotinamide (di-hydro-MNA), and tested its effect on the levels of 1-MNA and choline in brain extracellular fluid. Administration of di-hydro-MNA (1 mmol/kg s.c.) caused 4- and 9-fold increases in plasma and liver 1-MNA levels, respectively, as determined by HPLC. Concomitantly, the brain tissue levels of 1-MNA were increased by a factor of twenty. In brain extracellular fluid, the injection of di-hydro-MNA (1-3 mmol/kg s.c.) elevated 1-MNA levels by 3- to 10-fold to maximum levels of >10 μM.

Perioli et al. (in “Potential prodrugs of non-steroidal anti-inflammatory agents for targeted drug delivery to the CNS”, Eur. J. Med. Chem. 2004, 39 (8):715-27) synthesized new potential prodrugs of several NSAIDs. These novel NSAID precursors were developed in order to increase the NSAID's access to the brain. In Perioli's molecules, the carboxylate group of the subject NSAIDs were attached to 1,4-dihydro-1-methylpyridine-3-carboxylate moities via an amino alcohol bridge. These modifications increased the lipophilicity of the NSAIDs beyond their base values. As is well appreciated in the art, lipophilicity is a critical criterion for oral uptake of drugs. Thus the use of 1,4-dihydro-N-methyl-nicotinamide, a compound that is more lipophilic than 1-MNA, provides a likely means of increasing blood levels of 1-MNA and achieving target pharmacological endpoints in the clinic.

Another approach to elevating blood levels of 1-MNA can be achieved by blocking catabolic paths that lead to 1-MNA breakdown. The principal metabolic pathway involving modification of 1-MNA involves the enzyme aldehyde oxidase (Obach et al., 2004, “Human liver aldehyde oxidase: inhibition by 239 drugs.” J. Clin. Pharmacol. 44 (1):7-19.). Aldehyde oxidase catalyzes the addition of water to the 2- and 4-positions of the pyridine ring, followed by oxidation of the resultant hydroxyl group. Oxidation of hydroxylated 1-MNA requires the loss of the equivalent of one H₂ molecule, namely two protons and two electrons.

In principle, metabolism of 1-MNA may be suppressed by the presence of an alkyl group (such as, for purposes of illustration only, CH₃) or a deuterium atom at either or both the 2 or 4- positions of 1-MNA. Without wishing to be bound to any particular theory, it is believed that deuterium-labeled or alkylated 1-MNA isomers may be metabolized far slower relative to parent 1-MNA and produce a steady-state concentration of 1-MNA in excess of what may be achieved with the all protium parent.

Deuterium analogs of the dihydro-species are also considered as useful with respect to elevating 1-MNA levels, as di-hydro-MNA is metabolized to 1-MNA in vivo. Structures containing a mono, di, or tri-deuteromethyl group (i.e., —CHₓD_{y} wherein x and y are independently 0, 1, 2 or 3, and x+y=3) instead of CH₃ at position 1, are also disclosed.

### Definitions

These and other embodiments of the invention will be described with reference to following definitions that, for convenience, are collected here.

The articles “a” and “an” are used herein to refer to one or to more than one (i.e. to at least one) of the grammatical object of the article. By way of example, “an element” means one element or more than one element.

The term “treatment” or “treating,” as used herein, is defined as the application or administration of a therapeutic agent, i.e., a compound of the invention (alone or in combination with one or more additional compounds), to a subject, or application or administration of a therapeutic agent to an isolated tissue or cell line from a subject (e.g., for diagnosis or ex vivo applications), who has a disease or disorder treatable with a compound of the invention, with the purpose to cure, heal, alleviate, relieve, alter, remedy, ameliorate, improve or affect the disease or disorder, or the symptoms of the disease or disorder. Such treatments may be specifically tailored or modified, based on knowledge obtained from the field of pharmacogenomics.

The term “subject” includes living organisms in which diseases or disorders treatable with the compounds of the invention can occur. The term “subject” includes animals, such as mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as chickens, ducks, geese, and transgenic species thereof; and cells, e.g., immortalized or nonimmortalized cells, derived therefrom.

Administration of the compositions of the present invention to a subject to be treated may be carried out using known procedures, at dosages and for periods of time effective to treat the disease or disorder treatable with a compound of the invention (such as a lipoprotein abnormality) in the subject. An effective amount of the therapeutic compound necessary to achieve a therapeutic effect may vary according to factors such as the state of the disease or disorder in the subject, the age, sex, and weight of the subject, and the ability of the therapeutic compound to inhibit the disease or disorder in question (such as a lipoprotein abnormality) in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for the therapeutic compound of the invention is between 1 and 500 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound of the present invention employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration may affect what constitutes an effective amount. The therapeutic formulations may be administered to the subject either prior to or after the onset of a disease or disorder (such as a lipoprotein abnormality). Further, several divided dosages, as well as staggered dosages, may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disorder or disease (such as a lipoprotein abnormality) in subjects.

### Compounds of the Invention

Compounds of the invention include compounds according to Formulas (I) and (II):

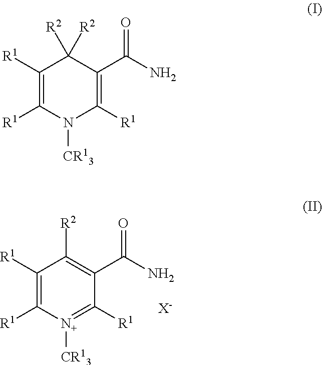

wherein

each R¹ is independently selected at each occurrence from the group consisting of H or D;

R² is independently selected at each occurrence from the group consisting of H, D, or CR¹₃, provided that where R² may be substituted on a molecule twice, both occurrences are not CR¹₃;

at least one of R¹ or R² is D; and

X⁻, when present, is a pharmaceutically acceptable counter ion, such that a pharmaceutically acceptable salt is formed.

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

1-methyl-nicotinamide , or a derivative, modification, or combination thereof, for administration may be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

### Formulations for Administration

In another embodiment, the present invention is directed to a packaged pharmaceutical composition comprising a container holding a therapeutically effective amount of a compound of the invention and instructions for using the compound to treat, prevent, or reduce one or more symptoms of one or more diseases or disorders treatable with a compound of the invention in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product.

Another embodiment of the invention is a pharmaceutical composition comprising a therapeutically effective amount of 1-methyl-nicotinamide, or an analog, derivative, modification, or combination thereof, and a pharmaceutically acceptable carrier.

The language “therapeutically effective amount” describes the amount of a given compound of the invention, or combinations of one or more compounds of the invention, that are effective to treat one or more diseases or disorders in a subject.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms mayn be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

A compound of the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For example, for oral administration, a compound of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating,systems available from Colorcon, West Point, Pa. (e.g., OPADRY™ Type, OY-C Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For parenteral administration, a compound of the invention for use in the method of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

With regard to transurethal administration, the formulation mayn comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer may be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit may be fabricated to disintegrate rapidly or over a period of several hours. The time period for complete disintegration may be in the range of from about 10 minutes to about 6 hours, e.g., less than about 3 hours.

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours.

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent may be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers may also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, 21^{st} Ed., Philadelphia, Pa., Lippincott Williams & Wilkins, 2005). Other ingredients, such as preservatives, colorants, lubricating or viscous mineral or vegetable oils, perfumes, natural or synthetic plant extracts such as aromatic oils, and humectants and viscosity enhancers such as, e.g., glycerol, may also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation may also comprise dry powder formulations, particularly insufflations in which the powder has an average particle size of from about 0.1 μm to about 50 μm, e.g., from about 1 μm to about 25 μm.

Topical formulations may be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

1-methyl-nicotinamide, or a derivative, modification, or combination thereof, of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

One common system utilized for intrathecal administration is the APT Intrathecal treatment system available from Medtronic, Inc. (Minneapolis, Minn.). APT Intrathecal uses a small pump that is surgically placed under the skin of the abdomen to deliver medication directly into the intrathecal space. The medication is delivered through a small tube called a catheter that is also surgically placed. The medication may then be administered directly to cells in the spinal cord involved in conveying sensory and motor signals associated with lower urinary tract disorders.

The term intravesical administration is used herein in its conventional sense to mean delivery of a drug directly into the bladder. Suitable methods for intravesical administration may be found, for example, in U.S. Pat. Nos. 6,207,180 and 6,039,967.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in US Patent Application Serial No. 20030147952, 20030104062, 20030104053, 20030044466, 20030039688, and 20020051820. Additional dosage forms of this invention also include dosage forms as described in PCT Patent Application Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles for example, by injection or in the form of wafers or discs by implantation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

The therapeutically effective amount or dose of a compound of the present invention will depend on the age, sex and weight of the patient, the current medical condition of the patient and the nature of the disorder or disease (such as lipoprotein abnormalities) being treated. The skilled artisan will be able to determine appropriate dosages depending on these and other factors. A suitable dose of a compound of the present invention may be in the range of from about 500 mg to about 10,000 mg per day, such as from about 750 mg to about 3000 mg, for example, from about 50 mg to about 1000 mg, such as about 75 mg to about 750 mg per day. The dose can be administered in a single dosage or in multiple dosages, for example from 1 to 4 or more times per day. When multiple dosages are used, the amount of each dosage can be the same or different. For example, a dose of 100 mg per day may be administered as two 50 mg doses, with about a 12 hour interval between doses.

It is understood that the amount of compound dosed per day may be administered every day, every other day, every 2 days, every 3 days, every 4 days, every 5 days, etc. For example, with every other day administration, a 500 mg per day dose may be initiated on Monday with a first subsequent 500 mg per day dose administered on Wednesday, a second subsequent 500 mg per day dose administered on Friday, etc.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein including prodrug forms. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

The contents of all references, issued patents, and published patent applications cited throughout this application are hereby expressly incorporated by reference in their entireties. It should be understood that the use of any of the compounds described herein are within the scope of the present invention and are intended to be encompassed by the present invention and are expressly incorporated herein for all purposes.

What is claimed:

1. A composition comprising at least one compound of Formula (I) or Formula (II): 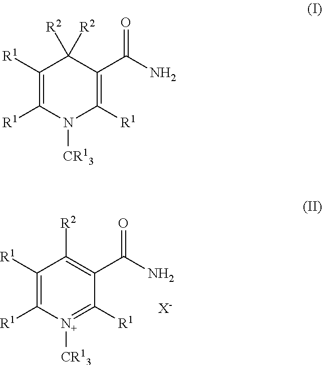
wherein
each R¹ is independently selected at each occurrence from the group consisting of H or D;
R² is independently selected at each occurrence from the group consisting of H, D, or CR¹₃, provided that where R² may be substituted on a molecule twice, both occurrences are not CR¹₃;
at least one of R¹ or R² is D; and,
X⁻, when present, is a pharmaceutically acceptable counter ion.

2. The compound of claim 1, wherein X⁻ is selected from the group consisting of chloride, bromide, benzoate, salicylate, acetate, citrate, and lactate.

3. A method of treating or diagnosing a disease or disorder in a subject in need thereof, comprising administering to said subject a therapeutically effective amount of a composition comprising at least one compound of Formula (I) or Formula (II): 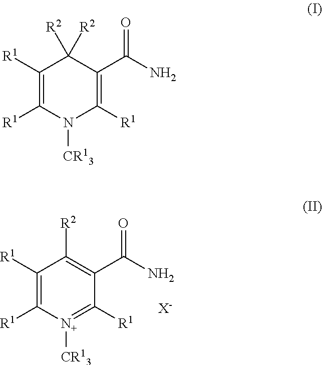
wherein
each R¹ is independently selected at each occurrence from the group consisting of H or D;
R² is independently selected at each occurrence from the group consisting of H, D, or CR¹₃, provided that where R² may be substituted on a molecule twice, both occurrences are not CR¹₃;
at least one of R¹ or R² is D; and,
X⁻, when present, is a pharmaceutically acceptable counter ion.

4. The method of claim 3, wherein said disease or disorder is heart disease, diabetes, cancer, osteoporosis, obesity, skin disorders, venous thrombosis, myocardial infarction, stroke, congestive heart failure, Alzheimer's disease, eczema, atherosclerosis, hyperlipidemia, hypertension, cerebral vasospasm, coronary vasospasm, bronchial asthma, preterm labor, erectile disfunction, glaucoma, vascular smooth muscle cell proliferation, myocardial hypertrophy, malignoma, ischemia/reperfusion-induced injury, endothelial dysfunction, Crohn's Disease, colitis, irritable bowel disease, neurite outgrowth, Raynaud's Disease, angina, or benign prostatic hyperplasia.

5. The method of claim 3, wherein said at least one compound of Formula (I) or Formula (II) is administered orally, nasally, rectally, intravaginally, intravesically, parenterally, buccally, sublingually, or topically.

6. The method of claim 3, wherein said at least one compound of Formula (I) or Formula (II) is formulated using one or more pharmaceutically acceptable excipient chosen from the group consisting of starch, sugar, cellulose, diluent, granulating agent, lubricant, binder, disintegrating agent, wetting agent, emulsifier, coloring agent, release agent, coating agent, sweeting agent, flavoring agent, perfuming agent, preservative, antioxidant, plasticizer, gelling agent, thickener, hardener, setting agent, suspending agent, surfactant, humectant, carrier, and stabilizer, or a combination there.

7. The method of claim 3, wherein said subject is a mammal.

8. The method of claim 7, wherein said subject is human.
